(19) United States
(12) Patent Application Publication
(10) Pub. No.: US 2014/0378859 A1
(43) Pub. Date: Dec. 25, 2014

(54) Method of Multichannel Galvanic Skin Response Detection for Improving Measurement Accuracy and Noise/Artifact Rejection

(71) Applicants: Alexander Taratorin, Palo Alto, CA (US); Serguei Pantchenko, Scotts Valley, CA (US)

(72) Inventors: Alexander Taratorin, Palo Alto, CA (US); Serguei Pantchenko, Scotts Valley, CA (US)

(21) Appl. No.: 13/927,085

(22) Filed: Jun. 25, 2013

(51) Int. Cl. A61B 5/053; A61B 5/00

(52) U.S. Cl. CPC A61B 5/0533; A61B 5/7203
USPC 600/547

(57) ABSTRACT

Proposed is a method of multichannel GSR detection for improving measurement accuracy and noise/artifact rejection in measurement of the galvanic skin response on the skin of an individual. The method comprises multichannel GSR signal detection with at least two electrode pairs that are attached to a body of an individual in contact with the skin in different locations, wherein the GSR signals are measured and recorded while the level of excitation of the nervous system of the individual varies. The method is based on the fact that only events that show high temporal correlation in two or more signal registration channels are considered to be physiological GSR reactions. Time-domain correlation allows not only improved detection of high-amplitude events but also improved sensitivity to smaller events that were ignored in standard bipolar GSR recording.

### BACKGROUND OF THE INVENTION

1. Field of the Invention

This invention relates in general to a method for galvanic skin response (GSR) measurements, and, more particularly, to a method of multichannel GSR detection for improving measurement accuracy and noise/artifact rejection in such measurements.

2. Description of the Related Art

The GSR (galvanic skin response) or electro-dermal response (EDR) is a well-known phenomenon in physiology. Changes in electrical skin resistance were discovered in the early 1900s. These changes are usually detected by using low DC current applied to the skin region between two electrodes and measuring skin resistance.

In the early 1930s it was established that changes in skin resistance constitute a sequence of short pulses and show strong correlation to a person's physiological and psychological states, such as emotions, levels of physical and mental activity, responses to external stimuli (visual, audio, pain, etc.). A comprehensive description of skin electro-dermal activity and its mechanisms can be found in Bloelectromagnetism: Principles and Applications of Bioelectric and Biomagnetic Fields, by Jaakko Malmivuo and Robert Plonsey; Oxford University Press, New York, 1995.

The mechanisms of GSR have been extensively studied in numerous publications and are associated with the activity of sweat glands, which is not always directly related to sweat secretion and which is the primary mechanism of thermal regulation. Rather, the excretory parts of the sweat glands are controlled by the sympathetic nervous system (see, for example, V. V. Dementienko, V. B. Dorokhov, et al, “Hypothesis about the nature of electrodermal reactions,” Journal of Psychophysiology, 1988, v. 30, 1, p. 267). Numerous publications demonstrate the strong correlation of GSR signals to human emotions and cognitive tasks (see, e.g., N. Nourbakhsh, Y. Wang. Using Galvanic Skin Response for Cognitive Load Measurements in Arithmetic and Reading Tasks, Proceedings of 24th Australian Computer-Human Interaction Conference), pain response level (H. Boyce, S. Loughrian. Skin Conductance as a Measure of Pain and Stress in Hospitalized Infants,” Human Early Development, 2006, 82, pp. 603-608, 2006), response to advertising (R. Ohme, D. Rekowska. Analysis of Neurophysiologic Reactions to Advertising Stimuli by Means of EEG and Galvanic Skin Response Measures, Journal of Neuroscience, Psychology and Economics, 2009, vol. 2, 1, pp. 21-31), etc. Other applications include lie detectors, monitoring of GSR as a response to music, and controlling the level of operator activity, such as determining the level of attention and controlling (warning) the operator's falling asleep.

There are many commercial systems, including systems of electrodes and amplifiers for GSR measurements. For example, commercially available systems for physiology and lab research are offered by Psychlab, iWorx (GSR-2000), and many others.

While GSR instrumentation is relatively simple, most available systems are not well suited to practical use and are applicable mostly under laboratory conditions because special gel electrodes are required in order to provide good skin contact. Changes in skin-electrode contact caused by movement, sweating, etc. result in a high level of noise and artifacts that may look similar to the GSR signal. Therefore, in practice it is difficult to achieve reliable detection of physiological GSR signals.

A significant advancement in the field of GSR signal detection is disclosed in U.S. Pat. No. 6,167,299 issued in 2000 to Galchenkov, et al. In this patent it is proposed to use GSR signal detection based on the specific shape of a GSR reaction, having a fast rising edge and a slower falling edge. In order to compensate for slow trends and changes in overall skin resistance, it is proposed to analyze derivatives of the logarithm of an electrode signal. Each signal pulse/spike detected in the incoming sequence of GSR signals is analyzed in terms of first and second derivatives, and the decision whether a pulse signal can be attributed to a physiological reaction or a contact artifact is made based on peaks and time intervals of derivative changes. The authors of U.S. Pat. No. 6,167,299 also propose a hardware-based solution for signal detection that includes the use of signal logarithmic converters, differentiators, and comparators. This method allows for improved GSR signal detection, which is less sensitive to motion artifacts. The detection method disclosed in U.S. Pat. No. 6,167,299 is used in commercially available systems such as Vigiton® (Driver Vigilance Telemetric Control System.) This system consists of a bracelet and ring type of electrodes that transmit GSR signals to a control unit. If the driver is starting to fall asleep, the frequency of GSR events decreases, and the control unit generates a warning signal, keeping the driver awake. Similar principles have been used for a railroad engine driver vigilance telemetric control system (EDVTCS).

A number of US Patent publications disclose different aspects of GSR. For example, U.S. Pat. No. 8,396,530 issued to Wilder-Smith, et al, in 2013 for a Method for Biosensor Usage with Pressure Compensation discloses the use of an additional pressure sensor that changes the degree of skin contact with electrodes for improved detection of GSR signals. International Patent Publication WO2013044185A3 (inventors, Picard Rosalind Wright, et al, published in 2013) that relates to “Clinical Analysis Using Electrodermal Activity discloses a method for capturing electro-dermal reaction data with subsequent transmission to a web service for consequent analysis. Another international Patent Publication WO2006106408A3 for System and Method for Estimation of Human Activity and Mood States Based on Rapid Eye Movements and Galvanic Skin Response (inventors, Serguei Bedziouk, Yurij Ya Golikov, and Anatoly N. Kostin; published in 2006) discloses a combination of GSR and eye movement measurements for improved detection of physiological responses.

However, the basic method for GSR measurement disclosed in available publications and patents does not fully resolve the fundamental problem of artifacts caused by changes in electrode-skin contact. It is an object of this invention to provide an improved method for GSR detection that provides higher accuracy and reliability for physiological research and practical applications.

### SUMMARY OF THE INVENTION

In order to provide reliable discrimination of GSR signals from noise and contact/motion-related artifacts, it is proposed to use multiple (two or more) electrode pairs, preferably in symmetric positions, e.g., on two different arms. The invention is based on the fact that the GSR signals registered in response to physiological reactions of the central nervous system are synchronous, whereas noise and motion artifacts that accompany the recorded GSR signals are not correlated. Examples of such an excitation event may comprise, e.g., an unexpected loud sound, an unexpected obstacle in the path of a driver, a confusing question, occurrence of a pain, etc. By comparing time dynamics of recorded GSR signals, only events that show high temporal correlation in two or more signal registration channels are considered to be physiological GSR reactions. Time-domain correlation allows not only improved detection of high-amplitude events but also improved sensitivity to smaller events that were ignored in standard bipolar GSR recording. Both the foregoing and the following descriptions are exemplary and explanatory only and are not intended to limit the claimed invention or application thereof in any manner whatsoever.

### BRIEF DESCRIPTION OF THE DRAWINGS

The accompanying drawings, which are incorporated in and constitute a part of this specification, exemplify the embodiments of the present invention and, together with the description, explain and illustrate principles of the inventive technique.

FIG. 1 is a graph illustrating a noisy raw GSR electrode signal (thin lines) and GSR signals obtained after low-pass filtering (thick line).

FIG. 2A is a graph that shows raw GSR electrode signals before trend and baseline correction.

FIG. 2B is a graph that shows the same signals as in FIG. 2A after trend and baseline correction using digital filtering.

FIG. 3A is a graph that shows GSR signals from three electrodes.

FIG. 3B shows local standard deviation/activity level of the signals in FIG. 3A.

FIG. 3C is a graph that shows GSR signals illustrating the activation (eyes open) and relaxation (eyes closed) sequences, where relaxation intervals are circled, and the signals being shown with trends are removed.

FIG. 3D is a time-domain local activity plot for the same signals as shown in FIG. 3C estimated as a standard deviation of signals taken at 3 s intervals.

FIG. 3E is a graph that illustrates a region of relaxation (eyes closed) showing a slowly decreasing pattern of low-amplitude GSR activity.

FIG. 3F is a graph with the same signals as in FIG. 3E but estimated as a standard deviation of the signals.

FIG. 4A shows an electrode pair of the invention and an insulating ring to be placed between the electrodes.

FIG. 4B is an example of placement of the electrode pairs on the fingers of the same hand.

FIG. 5 is an example of a system suitable for implementation of the method of the present invention for acquisition and processing recorded GSR signals.

### DETAILED DESCRIPTION

The method of the present invention is based on the principles described below.

It already has been established in numerous studies that GSR activity level strongly correlates with human activity level, emotional state, pain, stress, and other physiological or psychological changes. Since the origin of GSR is attributed to the central nervous system, it is expected that similar sweat-gland reactions occur in different parts of the body at the same time.

It is well known that sweat-gland distribution is not uniform across the human body. The highest density of sweat glands is observed in the hand palmar areas (over 500 sweat glands per 1 cm²), fingertips, and foot soles. Other regions have lower densities of sweat glands and therefore show lower amplitudes of GSR signals.

The GSR signal detected by an electrode effectively represents the spatial average of individual glands activity under the electrode. Therefore, high-amplitude GSR events correspond to synchronous activation of a plurality of sweat glands.

There are very few detailed studies on spatial and temporal sweat-gland activity. The paper by T. Nishiyama, et al, Irregular Activation of Individual Sweat Glands in Human Sole Observed by Videomicroscopy, Autonomous Neuroscience, Apr. 12, 2001, pp. 117-126) is one of the most detailed scientific investigations of individual gland activity. The main conclusion of the T. Nishyama study is that an ensemble of a large number of sweat glands typically exhibits simultaneous activation.

The invention is based on the fact that physiological responses caused by activation of the central nervous system in response to a certain excitation event are synchronous, whereas noise and motion artifacts that accompany the recorded GSR signals are not correlated. Examples of such an excitation event may comprise, e.g., an unexpected loud sound, an unexpected obstacle in the path of a driver, a confusing question, occurrence of pain, stress, anger, etc. By comparing time dynamics of recorded GSR signals, only events that show high temporal correlation in two or more signal registration channels are considered to be physiological GSR signals. Time-domain correlation allows not only improved detection of high-amplitude events but also improved sensitivity to smaller events that are difficult to reveal in standard bipolar GSR recording. In other words, the results of GSR measurements produce synchronous GSR signals and nonsynchronous GSR signals. Based on the above information, the method of the invention comprises the following steps: (1) providing multichannel signal detection with at least two electrode pairs for at least two-channel GSR measurements; (2) attaching these at least two electrode pairs in contact with the skin in different locations on the body of an individual; (3) applying current to the electrodes and measuring and recording the GSR signals simultaneously through said at least two channels in said different locations in different physiological and/psychological states of the individual to obtain recorded data of the GSR while the level of excitation of the nervous system of the individual varies; (4) revealing from the recorded data the noise/artifact signals as data that are not synchronized in said different locations; and removing the nonsynchronized GSR signals from the recorded data, thus leaving only those data that relate to the GSR signals.

The step of removing the nonsynchronous GSR signals comprises ignoring the signals that are nonsynchronous in at least one channel while accounting for synchronous signals in at least two channels.

It is preferable that the aforementioned different locations be on different parts of the body and in symmetrical positions, e.g., on the left hand and on the right hand.

As another possible embodiment, the electrode locations could be asymmetrical. For example, one electrode pair may be fitted on the fingers of the hand and another electrode pair may be attached to the sole of the foot of the individual. In this case, GSR recorded from different asymmetrical body locations may exhibit certain timing delays, which may be accounted for during subsequent data processing.

The method further comprises a step of improving the accuracy of GSR measurement for real-time detection by processing and analyzing the recorded data with use of a data processing method selected from the group consisting of: (a) low-pass filtering of signals in order to remove high-frequency noise; (b) compensating a low-frequency signal trend and differences in DC resistance; (c) detecting GSR signals in each channel by identifying individual peaks of the signal; and (d) calculating the time domain of GSR information if the results of GSR registration are used to cause a feedback signal to the individual or to an electronic system.

The feedback signal mentioned in aforementioned item (d) may comprise, e.g., a warning signal to a driver when the driver shows signs of drowsiness, or any other signals that are to be activated under conditions critical for an individual. Other examples of feedback signals are those used to control the emotional state of an individual who participates in activities such as computer games and physical exercises.

An event that innervates the nervous system of an individual may be a natural event caused by movements of the individual, mental activity such as reading, or an artificially generated event such as generation of unexpected noise, a sudden question, etc. The events of the latter type are used, e.g., in lie detectors.

As mentioned above, after the signal from each individual electrode pairs is acquired, it can be processed and analyzed using different methods of signal processing and analysis. This data processing can be performed by specialized electronic devices (e.g., low-pass filters, amplifiers, differentiators) and/or by digital signal processing algorithms performed by a dedicated computer or microprocessor. Modern electronics devices have powerful processing units and large memory capacity and therefore any type of processing functions can be performed in a dedicated microprocessing unit built into the signal acquisition device or equivalently by a dedicated mobile device application or a computer. Such applications are well known in prior art. This may be, e.g., a mobile phone application for analysis of physiological functions of the type described by D. Sahchez, T. Collins, et al, in Cellular Phone-based Biofeedback to Treat Physical and Mental Disorders, 2012 IEEE International Conference on Health Networking, Applications and Services; M. Poh, N. Swenson, R. Pickard in A Wearable Sensor for Unobtrusive, Long-term Assessment of Electrodermal Activity, IEEE Transactions on Biomedical Engineering, vol. 57, 5, 2010, pp. 1243-1251).

The primary goal of data processing is real-time detection of GSR. In order to detect GSR events from raw electrode resistance data, several very basic and well-known steps are typically required.

As mentioned above, one of the methods for processing and analyzing the GSR data is low-pass filtering of a signal in order to remove high-frequency noise. External noise originates from multiple sources: electronics, external signals from electrical and cellular networks, etc.

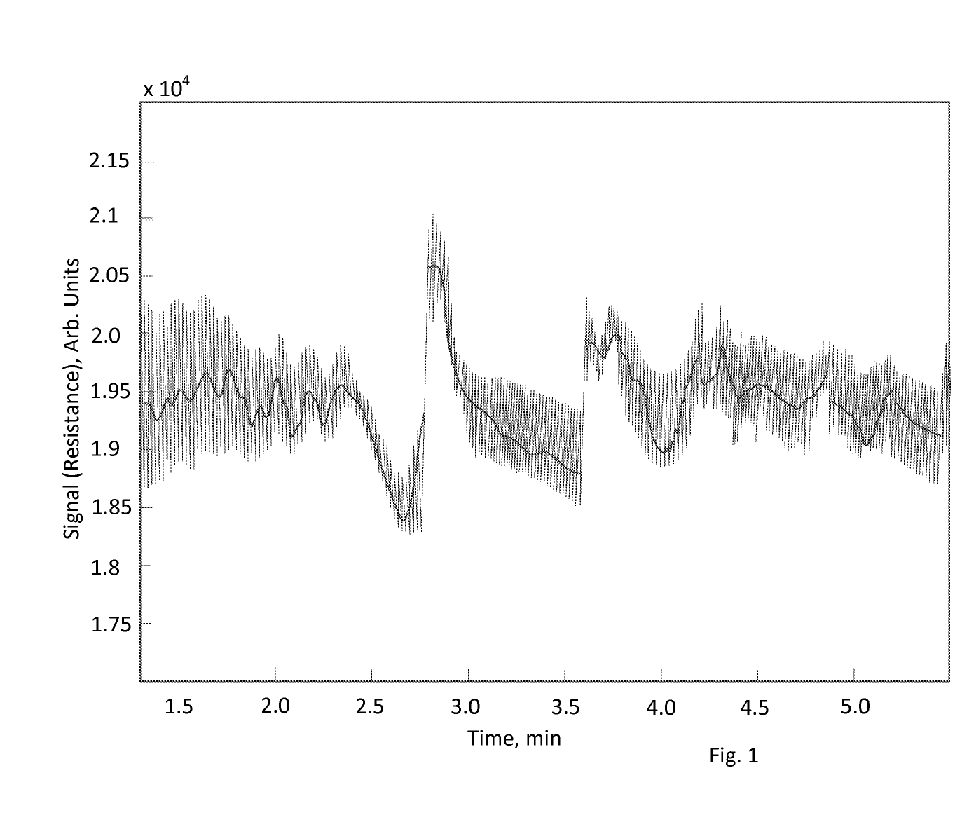

Low-pass filtering is performed using digital filtering, which is well known in prior art, e.g., A. Oppenheim, R. Schafer, and J. Buck, Discrete Time Signal Processing, Prentice Hall, 1975. FIG. 1 below illustrates an example of filtering a noisy GSR signal. A a noisy raw GSR electrode signal is shown by thin lines, and a GSR signals obtained after low-pass filtering and thus cleared from the high-frequency noise signal is shown by a thick line.

According to one or several aspects of the invention, the recorded GSR data can be processed and analyzed by compensating the low-frequency signal trend and the differences in DC resistance.

One of the methods suitable for processing data obtained by measuring GSR signals by the method of the invention consists of removing a noninformative average signal level, which can be caused by differences in skin conductivity in different body regions and does not carry GSR information. This operation can be performed by estimation of signal average value over a specified time interval (e.g., 1 min) and subtracting this average from the current signal sample. Simple implementation of this trend subtraction can be performed in hardware by using a low-pass filter and signal subtraction unit. However, this operation is equally simple to implement using raw samples by calculating and subtracting average value.

For additional detection of small events, the method may comprise a step of determining all synchronous GSR signals independent of the value of their amplitude and revealing these GSR signals as relevant to the physiological and/or psychological state.

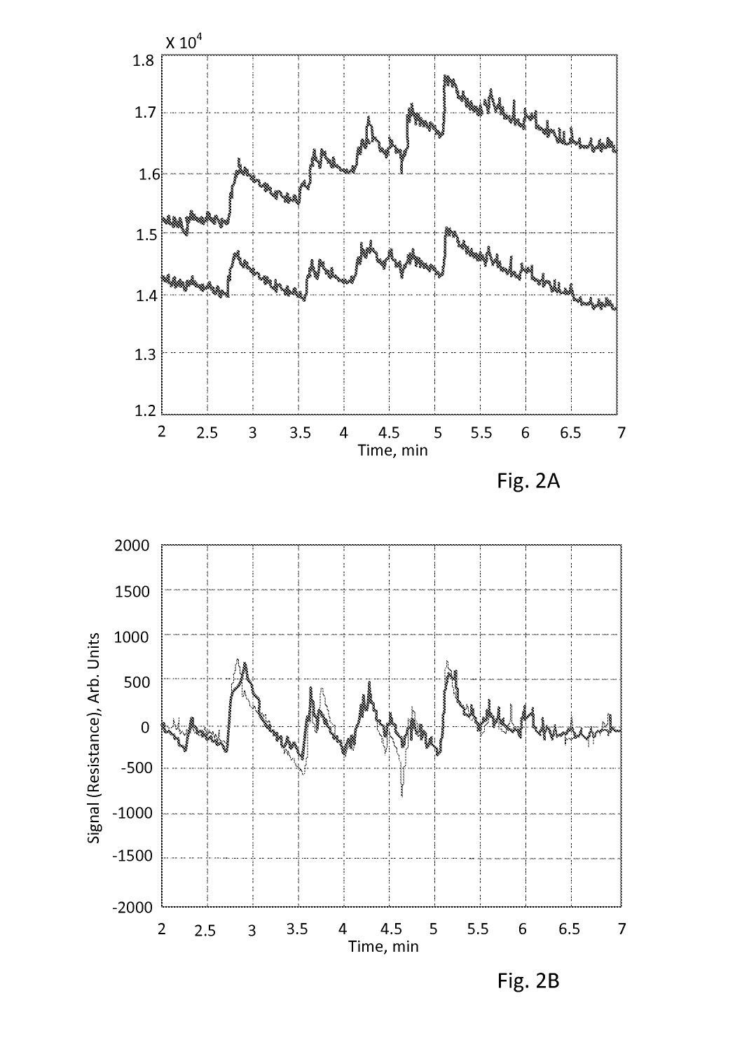

FIGS. 2A and 2B illustrate implementation of the method of the invention for measuring GSR signals by two electrode pairs in two different locations on the skin of the individual with the use of two electrode channels initially having different resistance levels and trends. Raw recordings (before processing) are shown in FIG. 2a, while signals after trend compensation with digital filtering are shown in FIG. 2b. As can be seen, signals obtained after drift compensation are simpler to compare and analyze.

Still another method suitable for processing and analyzing data is a GSR signal detection method for detecting physiologically related GSR activity and for rejecting motion and contact artifacts. Detection of GSR signals is described in multiple prior art publications, such as U.S. Pat. No. 6,167,299 (issued to Galchenkov, et al, in 2000, which is based on analysis of signal first and second derivative), and the paper by D. Bach, G. Flandin, et al, titled Time Series Analysis for Rapid Event-related Skin Conductance Responses, Journal of Neuroscience Methods, 184, 2009, pp. 224-234 and others.

According to the method of the invention, multichannel GSR signal detection does not necessarily require a priori knowledge and dedicated algorithms for GSR waveforms. Detection of physiological component of electrode signals can be done based merely on time correlation of signals recorded in two or more channels. This time domain detection can be based on several criteria, which are described in the following paragraph.

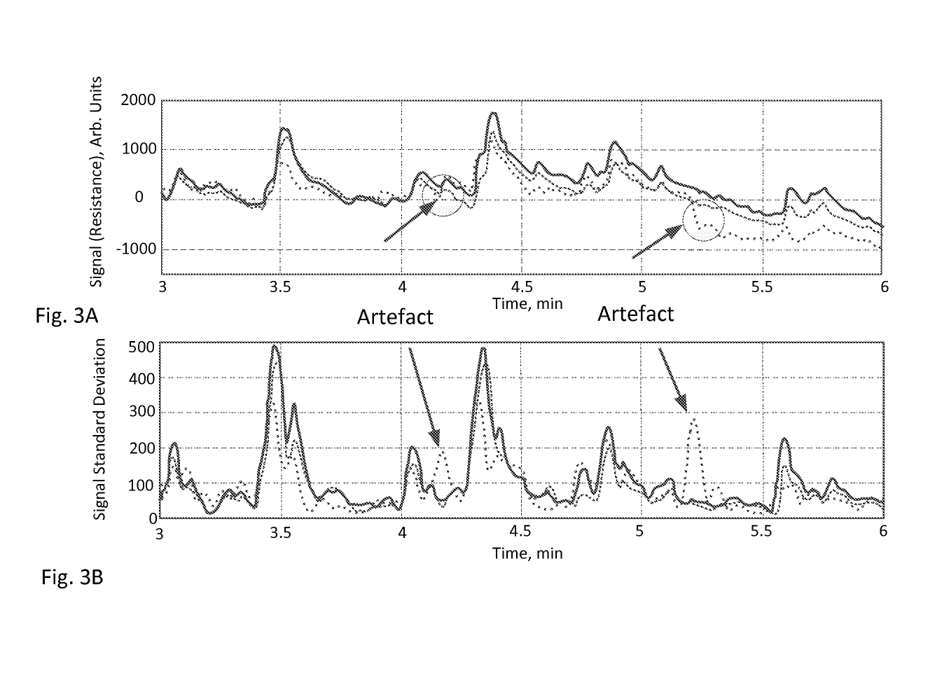

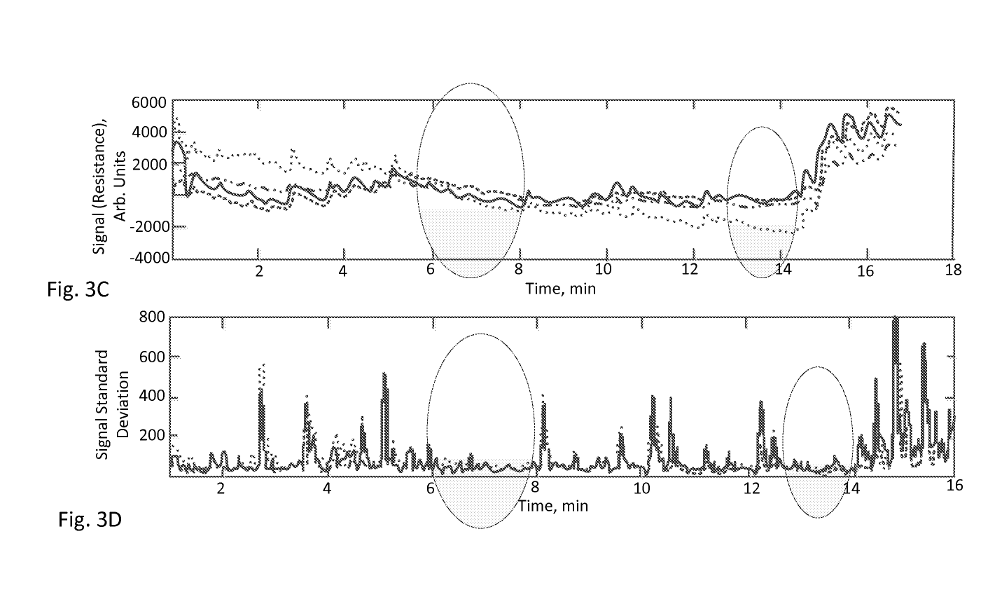

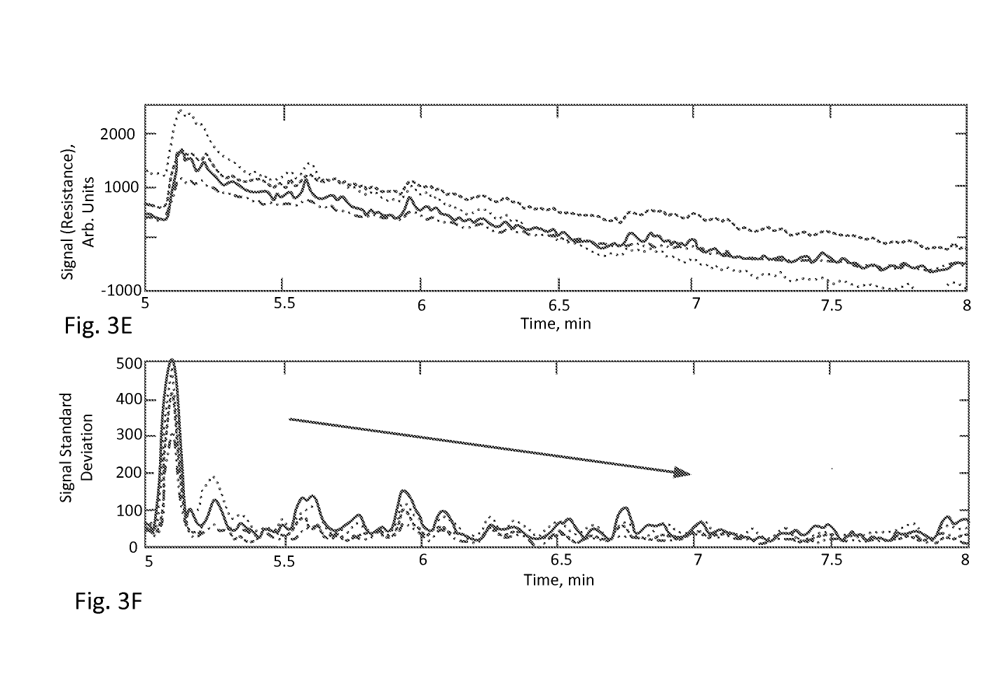

Simultaneous (synchronous) changes of local signal standard deviation, e.g., over a specified signal interval, e.g., during each 20-s interval. In this way we obtain a plurality of standard deviation values for defining local activities of skin response at given time instances. FIGS. 3A and 3B show signals recorded from three electrodes in different hands, wherein one signal is shown by a solid line trace, one signal is shown by a dense-dot line trace, and one signal is shown by a non-dense-dot line trace. The electrode signal shown by the non-dense-dot line trace results has intermittent skin contact changes that result from hand and finger motions. The arrows in FIG. 3A show these instances of change and represent raw electrode signals after low-pass filtering and trend removal, as described above.

At least two signals are needed because with signals from less than two electrode pairs, it is difficult to decide which of the signal changes shown in the dotted line traces correspond to GSR and motion artifacts. For example, the event at 5.2 min (right arrow) possibly can be detected by using methods based on signal derivatives described in U.S. Pat. No. 6,167,299. However, a small signal change that occurs at 4.2 min (left arrow) has the characteristic shape similar to the GSR response and can be mistaken for physiological GSR reaction. Comparison of local activity (signal standard deviation) in FIG. 3B, however, clearly reveals that peaks in channel activity shown by the non-dense-line are not correlated to activities shown by other channels, while all other events are synchronous.

Thus, the GSR signal detection in two channels can be based on rejection of all events that are not synchronous. Method accuracy can be improved using three or more channels for implementation of a simple “voting” principle. For example, if a certain event (a peak or minimum of local activity level) is detected in all but one channel, this channel is assumed as unrelated, but signals from all other channels are accepted.

Also, it should be noted that the method of synchronous event detection simultaneously occurring in different locations reveals finer details and low-value amplitude events in the GSR response, which could not be reliably detected in single-channel detection.

Detection of correlated local activity can be achieved with the use of different methods, e.g., by analyzing time derivatives (first and second) of peaks and zero crossings, time derivatives of standard deviation signals, location of standard deviation peaks and valleys, and local time cross-correlation. All of these signal analysis methods are well known in related art (see, e.g., A. Oppenheim, R. Schafer, and J. Buck, Discrete Time Signal Processing, Prentice Hall, 1999). Any combination of multichannel cross correlation with GSR signal detection described in the references cited above also applies to this approach. The next step of the method is calculating statistical parameters of the synchronous GSR signals and using these parameters for real-time characterization of physiological and psychological states of the individual and/or providing feedback signals to the individual or to an electronic system.

As mentioned above, in measurement of the GSR signals, the local activity of the individual is represented by parameters such as distribution of GSR peaks over a specified time interval, distribution of time intervals between adjacent peaks, and by changes in peak amplitudes and intervals distributed over the measurement period. Statistical parameters are calculated by a microprocessor unit and are usually represented as histograms. Depending on the specific application, different statistical measures are required. For example, for analysis of an operator state of attention (vigilance), information is collected about an average interval between strong GSR activity signals.

Typically 15 to 20-s intervals correspond to a normal activity level, and if there are no events during 45 to 60 s, this is an indication of reduced attention level, while absence of signals for 100 to 120 s indicates deep relaxation and can be considered a sign of dangerous loss of control.

As an example of activity level evaluation, the applicants recorded a sequence of “activation” (e.g., reading) and “relaxation” (eyes closed) events by combining mental activity with relaxation. In order to estimate local activity level, local standard deviations of each channel signal taken at 3-s intervals were recorded. The results are shown in FIGS. 3C and 3D, where FIG. 3C shows GSR signals recorded from four channels, and FIG. 3D shows results of local activity estimation. Here, different signals are shown by four different lines, i.e., a solid line, a dense-dot line, a non-dense dot line, and a dash-and-dot line.

It can be clearly seen that mental activity (reading, counting) as well as head movement are characterized by high-amplitude GSR events. At the same time, relaxation periods are characterized by reduced local activity level and a smaller number of GSR events. This distribution of GSR events is well known in prior art, however the multichannel recording also illustrates that GSR events are synchronous; thus, improved detection of these events is possible. A more detailed view of a reduced activity region at 5 to 8-min intervals shown in FIG. 3E illustrates an interesting feature: there is a clear trace of low-amplitude events in all channels demonstrating slowly decreasing amplitude over 5.5 to 7-min time intervals. FIG. 3F is a graph with the same signals as in FIG. 3E but estimated as a standard deviation of the signals. In other words, the method of the invention makes it possible to reveal relaxation of a signal and to capture signals that reflect small features of this relaxation.

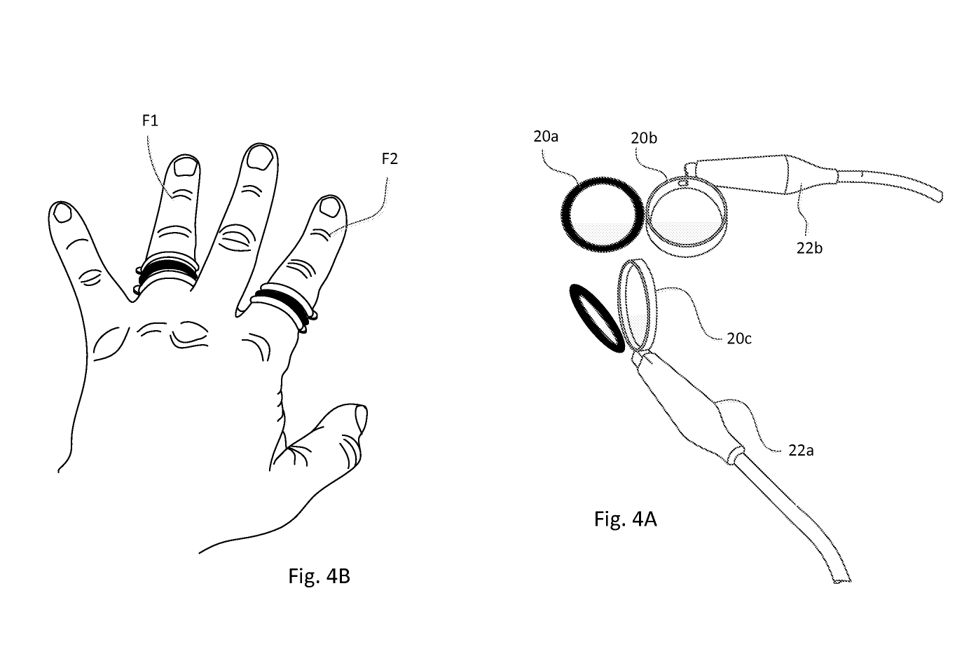

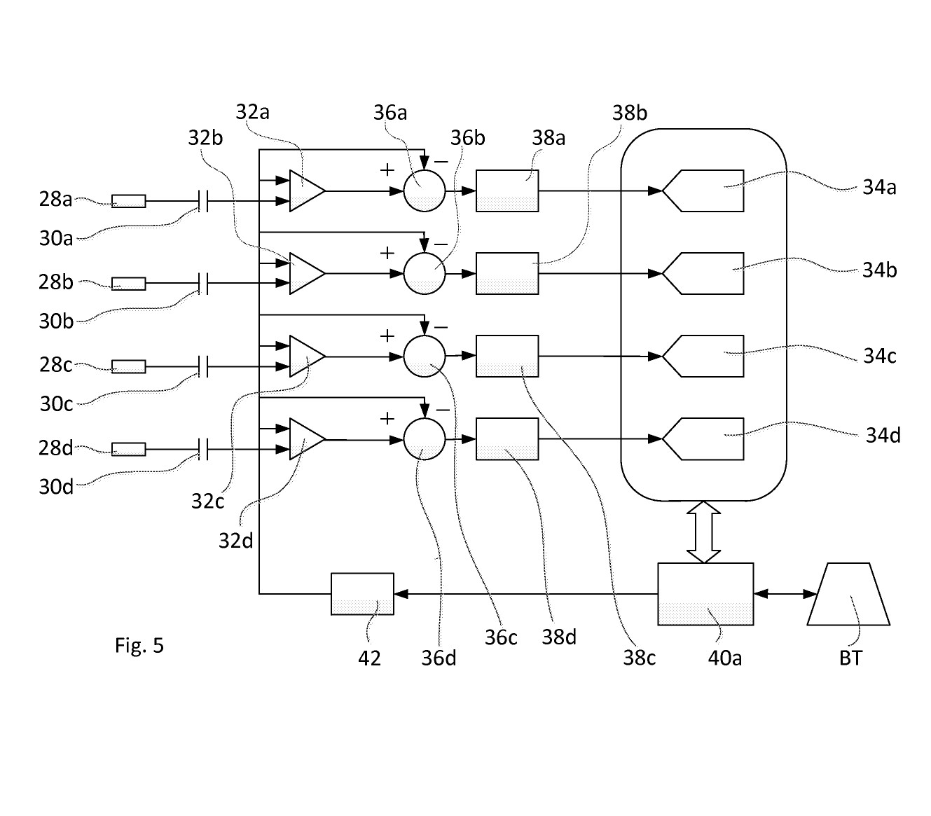

According to one or several aspects of the invention, each electrode pair comprises a pair of electrode elements electrically isolated from each other. Each such electrode pair is attached to different and preferably symmetrical parts of the body of the individual, e.g., to the left hand and to the right hand of the individual, respectively, in contact with the skin. Preferably, the electrode pairs are located in symmetrical positions on the left and right hands of the individual. This is required for providing synchronous excitation of the GSR signals under both electrode pairs. According to one or several aspects of the invention, as shown in FIGS. 4A and 4B, each electrode pair may comprise, e.g., a pair of metallic rings 20a and 20b and an insulating ring 20c between them, which is made, e.g., from rubber. The metallic rings 20a and 20b, which are electrically isolated from each other by the rubber ring 20c, are fitted on different fingers of the same hand. One metallic ring is grounded through a wire, e.g., a wire 22a, and the other metallic ring is electrically connected by a wire 22b to a signal acquisition and processing system 24, which is shown in FIG. 5 and is described below.

It is understood that the ring type of electrode elements are shown only as an example and that the designs, dimensions, and shapes of the electrodes may vary in a wide range, depending on the places where the electrodes are to be attached on the body of an individual for whom the method of the invention is to be implemented.

The data acquisition and processing system 24 suitable for implementation of the method of the invention is shown in FIG. 5 in a four-channel version and comprises four channels 26a, 26b, 26c, and 26d. Since all channels are identical, only one of them, i.e., the channel 26a, will be described hereinafter, but the identical components of other channels will be designated in FIG. 5 with the same reference numerals but with an addition of the letters “b”, “c”, and “d”, respectively. A four-channel system means that for ring-type electrode pairs, two electrode pairs are fitted to two fingers of the left hand and two other electrode pairs are fitted to the same fingers of the right hand.

Thus, the channel 26a comprises a respective electrode pair 28a, which is connected through an AC coupling 30a, e.g., a capacitor 30a, to a transimpedance amplifier 32a, which, in turn, is connected to an A/D converter through a subtractor 36a and a low-pass filter 38a. A function of the subtractor is to subtract an input sinusoidal signal and to leave only the useful signal. The low-pass filter 38a filters out all extraneous signals and leaves only signals on an operating frequency.

Different from the standard method of GSR measurements based on DC currents, the prototype system used for implementation of the proposed method uses low-frequency (200 Hz) AC sinusoidal current supplied to the skin between the ground and signal electrode elements. AC measurements of skin conductance are known in the literature (S. Grimmes, et al, Electrodermal Activity by DC Potential and AC Conductance Measured Simultaneously at the Same Skin Site, Skin Research and Technology, 2011, 17, pp. 26-34; and W. Bourchein, Publication Recommendations for Electrodermal Measurements, Psychophysiology, 49, 2012, pp. 1017-1034).

It is well known in prior art that AC measurements of skin conductance are different from DC methods due to a number of reasons. AC measurement reduces or completely eliminates electrode polarization, which occurs in the DC method, thus reducing signal drift. However, when AC current frequency is increased, current flow path changes and alternating current might penetrate the skin. Also, AC measurements measure total skin impedance, which depends not only on resistance but also on skin and electrode capacitance that may affect the shape of the GSR signal. For GSR it is recommended to use AC frequencies below 500 Hz.

In other words, the system controller 40a generates a 200-Hz square wave signal that is converted to a sinusoidal AC reference by the low-pass filter 42. This sinusoidal signal is supplied to four transimpedance amplifiers, which transmit this signal to each individual electrode pair (this transmission line is not shown). The transimpedance amplifiers 32a, 32b, etc., convert current into voltage signals that result in resistance measurement. As mentioned above, the sinusoidal signals are subtracted from the incoming signals and are filtered by the identical low-pass filters. Currently, with 200-Hz sinusoidal excitation, this corresponds to 100-Hz resistance measurement in each of four channels.

The respective A/D converters synchronously sample signals from the respective channels. The sampling occurs at the peaks of sinusoidal excitation, and samples corresponding to positive and negative peaks are subtracted, resulting in instantaneous resistance measurement from each channel. This synchronous sampling results in high signal-to-noise ratio and noise immunity. The controller 40a also communicates with a registering apparatus (not shown), e.g., via wireless communications, such as a Bluetooth® adapter, which transmits time series of resistance samples to a computer, mobile phone, or tablet for processing and analysis. This system can be “wearable” and is designed in a small form-factor to be placed inside a small watch-like bracelet.

Although the invention has been shown and described with reference to specific embodiments, it is understood that these embodiments should not be construed as limiting the areas of application of the invention and that any changes and modifications are possible, provided that these changes and modifications do not depart from the scope of the attached patent claims. For example, the number of electrode pairs may be different, e.g., from two or four, and the electrodes may be attached to parts of the individual's body other than the fingers on one or both hands, e.g., on the soles of the left and right feet. The data obtained in GSR measurements may be treated by methods different from those described in the specification. The electrode pairs, themselves, may have different configurations and dimensions, and the method of the invention may find application in many different fields where it is necessary to measure autonomic skin responses as psychological indicators.

Based on the above information, the method of the invention comprises the following steps: (1) providing multichannel signal detection with at least two electrode pairs for at least two-channel GSR measurements; (2) attaching these at least two electrode pairs in different locations on the body of an individual in contact with the skin; (3) applying a current to the electrode pairs and measuring and recording the GSR signals simultaneously through said at least two channels in said different locations in different physiological and/psychological states of the individual for obtaining recorded GSR data at different levels of excitation of the nervous system; (4) revealing from the recorded data the noise/artifact signals as data that are not synchronized in said different locations; and (5) removing the nonsynchronized GSR signals from the recorded data, thus leaving only those data that relate to GSR signals.

What we claim is:

1. A method of multichannel galvanic skin response detection for improving measurement accuracy and noise/artifact rejection in measurement of the galvanic skin response on the skin of an individual, the method comprising the following steps:
providing multichannel signal detection with at least two electrode pairs for at least two-channel GSR measurements;
attaching these at least two electrode pairs in different locations on the body of an individual in contact with the skin of the individual;
applying current to the electrode pairs and measuring and recording the GSR signals simultaneously through said at least two channels in said different locations in different physiological and/psychological states of the individual;
obtaining recorded GSR data while the level of excitation of the nervous system of the individual varies, the recorded GSR data containing synchronous GSR signals and nonsynchronous GSR signals;
revealing from the recorded data the noise/artifact signals as data that constitute nonsynchronous GSR signals in said different locations; and
removing the nonsynchronous GSR signals from the recorded data, thus leaving only those data that relate only to synchronous GSR signals.

2. The method of claim 1, wherein the level of excitation of the nervous system of the individual varies under the effect of events selected from natural events and artificially generated events.

3. The method of claim 2, wherein the different locations are locations on different parts of the body.

4. The method of claim 3, wherein the different locations are locations selected from symmetrical positions and asymmetrical positions on the body of the individual.

5. The method of claim 4, wherein the different locations are selected from the left and right hands and the left and right soles of the feet of the individual.

6. The method of claim 3, further comprising the step of calculating statistical parameters of the synchronous GSR signals and using these parameters for real-time characterization of physiological/psychological states of the individual and/or providing a feedback signal to the individual or to an electronic system.

7. The method of claim 4, further comprising the step of calculating statistical parameters of the synchronous GSR signals and using these parameters for real-time characterization of physiological/psychological states of the individual and/or providing a feedback signal to the individual or an electronic system.

8. The method of claim 5, further comprising the step of calculating statistical parameters of the synchronous GSR signals and using these parameters for real-time characterization of physiological/psychological states of the individual and/or providing a feedback signal to the individual or to an electronic system.

9. The method of claim 3, further comprising the step of determining all synchronous GSR signals independent of the value of their amplitude and revealing these GSR signals as relevant to the physiological and/or psychological states.

10. The method of claim 4, further comprising the step of determining all synchronous GSR signals independent of the value of their amplitude and revealing these GSR signals as relevant to the physiological and/or psychological states.

11. The method of claim 5, further comprising the step of determining all synchronous GSR signals independent of the value of their amplitude and revealing these GSR signals as relevant to the physiological and/or psychological states.

12. The method of claim 6, further comprising the step of determining all synchronous GSR signals independent of the value of their amplitude and revealing these GSR signals as relevant to the physiological and/or psychological states.

13. The method of claim 7, further comprising the step of determining all synchronous GSR signals independent of the value of their amplitude and revealing these GSR signals as relevant to the physiological and/or psychological states.

14. The method of claim 3, further comprising the step of calculating statistical parameters of the synchronous GSR signals for different GSR amplitudes and using these statistical parameters for characterization of the physiological/psychological states of the individual and/or for generating a feedback signal to be sent to the individual or to an electronic system.

15. The method of claim 5, further comprising the step of calculating statistical parameters of the synchronous GSR signals for different GSR amplitudes and using these statistical parameters for characterization of the physiological/psychological states of the individual and/or for generating a feedback signal to be sent to the individual or to an electronic system.

16. The method of claim 8, further comprising the step of calculating statistical parameters of the synchronous GSR signals for different GSR amplitudes and using these statistical parameters for characterization of the physiological/psychological states of the individual and/or for generating a feedback signal to be sent to the individual or to an electronic system.

17. The method of claim 3, wherein the step of removing the nonsynchronous GSR signals comprises ignoring signals that are nonsynchronous in at least one channel while accounting for synchronous signals in at least two channels.

18. The method of claim 5, wherein the step of removing the nonsynchronous GSR signals comprises ignoring signals that are nonsynchronous in at least one channel while accounting for synchronous signals in at least two channels.

19. The method of claim 8, wherein the step of removing the nonsynchronous GSR signals comprises ignoring signals that are nonsynchronous in at least one channel while accounting for synchronous signals in at least two channels.

20. The method of claim 16, wherein the step of removing the nonsynchronous GSR signals comprises ignoring signals that are nonsynchronous in at least one channel while accounting for synchronous signals in at least two channels.
